(19) United States
(12) Patent Application Publication
(10) Pub. No.: US 2013/0061853 A1
(43) Pub. Date: Mar. 14, 2013

(54) ENDOTRACHEAL TUBE STABILIZER APPARATUS AND METHOD

(71) Applicants: David Mark De lulio, Lafayette, LA (US); Donald Lee Morris, Littleton, CO (US); James Ringgold Strong, Clayton, MO (US); James Ringgold Strong, JR., St. Louis, MO (US); Jeffery F. Feng, Chesterfield, MO (US); Melvin Joshua Leedle, St. Louis, MO (US); Bryce George Rutter, St. Louis, MO (US)

(72) Inventors: David Mark De lulio, Lafayette, LA (US); Donald Lee Morris, Littleton, CO (US); James Ringgold Strong, Clayton, MO (US); James Ringgold Strong, JR., St. Louis, MO (US); Jeffery F. Feng, Chesterfield, MO (US); Melvin Joshua Leedle, St. Louis, MO (US); Bryce George Rutter, St. Louis, MO (US)

(73) Assignee: VCB IP Holdings, LLC, Clayton, MO (US)

(21) Appl. No.: 13/231,423

(22) Filed: Sep. 13, 2011

(51) Int. Cl. A61M 16/04; B23P 17/04

(52) U.S. Cl. 128/207.14; 29/428

(57) ABSTRACT

A neonatal endotracheal tube stabilizer has a tube cradle, a tube fixation element attached to said tube cradle and a stabilization bar having a plurality of engagement elements. The stabilization bar extends on either side of the tube cradle to engage two cheek pads. The cheek pads each have a releasable engagement interface dimensioned to engage the engagement elements of the stabilization rod in at least one engaged position. When so engaged, the cradle firmly holds the endotracheal tube in a position selected by a health care provider.

### RELATED APPLICATIONS

None

### BACKGROUND OF THE INVENTION

1. Field of the Invention

The invention is in the field of medical devices, particularly position and stabilization equipment for endotracheal tubes.

2. Related Art

Few things in medical care are as sensitive and demanding of precision for health care personnel as neonatal intubation. Proper placement of the newborn's “breathing tube,” ease, speed and accuracy of positioning, removal and replacement are of paramount importance. Accordingly, certain prior art devices have been developed to advance the art past simple taping of the tube to the newborn have been made. However, there remains a need in the art for devices improving ease, speed and accuracy of positioning, placement, removal and replacement of endotracheal tubes for neonates. There is a further need for devices that protect the skin of the child and for equipment that facilitates potential emergent response to changes in the child's status.

### SUMMARY OF THE INVENTION

The neonatal endotracheal tube stabilizer of the present invention has a tube cradle, a tube fixation element attached to said tube cradle and a stabilization bar having a plurality of engagement elements. The stabilization bar extends on either side of the tube cradle to engage two cheek pads. The cheek pads each have a releasable engagement interface dimensioned to engage the engagement elements of the stabilization rod in at least one engaged position. When so engaged, the cradle firmly holds the endotracheal tube in a position selected by a health care provider.

The engagement designs of both the tube cradle and between the stabilization rod and cheek pads provide for advantageous ease of use, flexibility of placement, and rapidity of release or readjustment.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

FIG. 1 is the exploded diagram of the endotracheal tube stabilizer of the present invention.

FIG. 2 is close up perspective view of a cheek pad.

FIG. 3 is a perspective view of the support bar and tube cradle of the present invention.

FIG. 4 is a top view of the support bar and tube cradle.

FIG. 5 is an isometric view of a part of an alternate embodiment.

FIG. 6 is an isometric view of a part of an alternate cheek pad embodiment.

FIG. 7 is an isometric view of a part of an alternate cheek pad embodiment.

FIG. 8 is an isometric view of a part of an alternate embodiment.

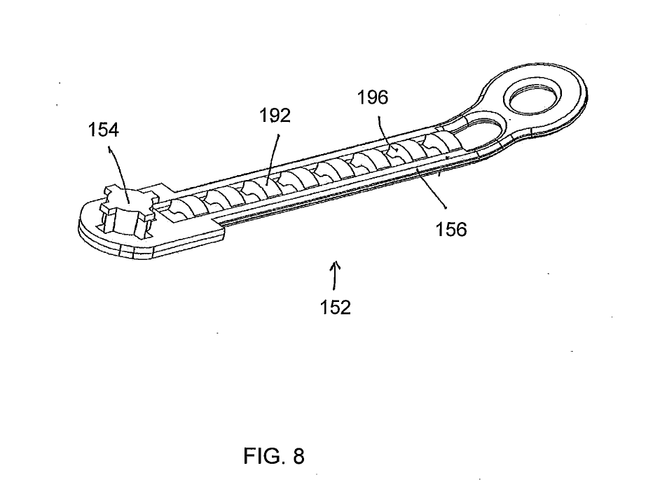

### DETAILED DESCRIPTION OF THE PREFERRED EMBODIMENTS

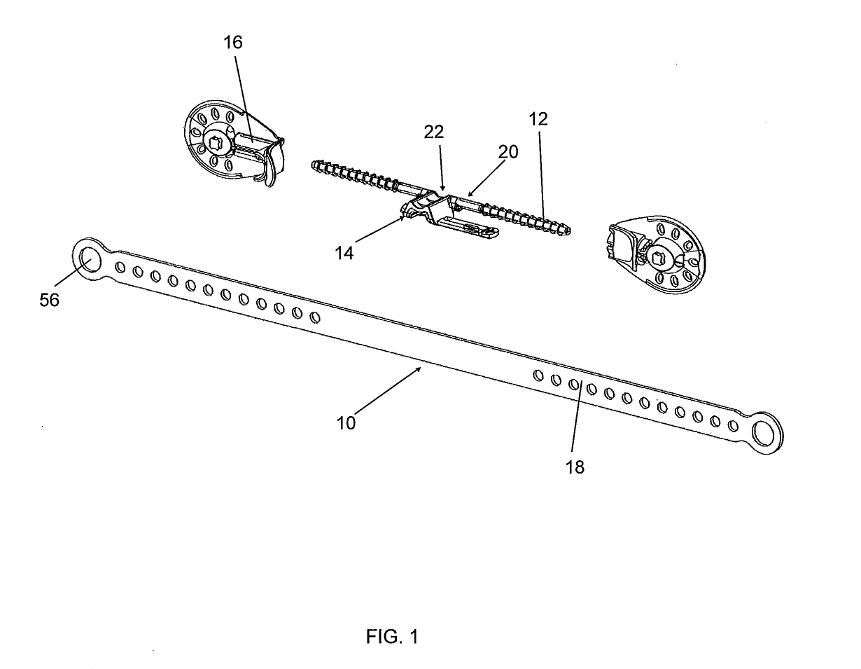

Referring now to the drawings in which like reference numbers indicate like elements, FIG. 1 is an exploded view of the neonatal endotracheal tube stabilizer of the present invention 10. The components of the invention include the support bar 12, the endotracheal tube (“ET Tube”) cradle 14, two cheek pads 16 and a neck strap 18.

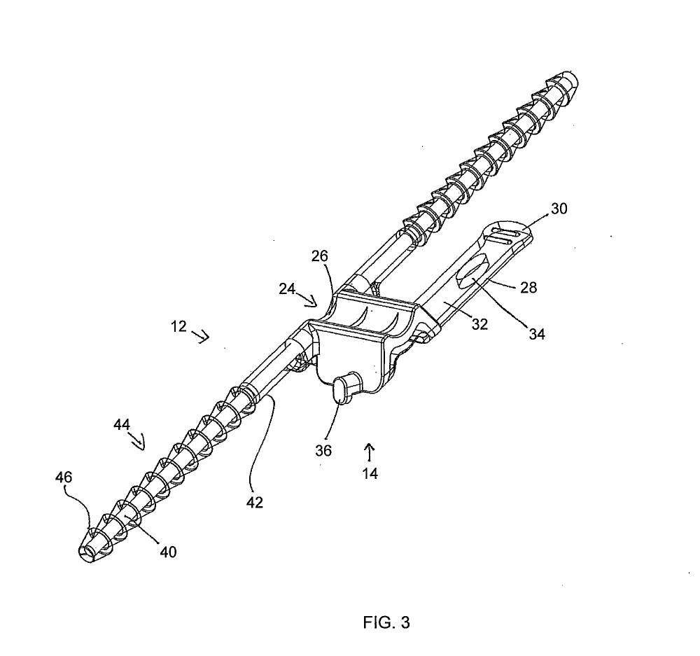

FIG. 3 depicts the support bar 12 and tube clamp 14 as assembled. The assembly of the ET Tube cradle 14 with the support bar 12 is by the engagement of ET Tube cradle slot 20 with central detent 22 in the support bar 12. Detent 22, as depicted, is substantially U-shaped and oriented downwards. This achieves simultaneously the effect of creating an appropriately dimensioned space for passage of the endotracheal tube, as well as an engagement with the ET Tube cradle 14 that is stable in multiple dimensions. Because the detent 22 is offset from the longitudinal axis of the support bar 12, which is oriented to be substantially transverse to the air passage or mouth of the patient, the detent 22 further promotes proper positioning of the ET Tube at the desired central position aligned with the patient's airway.

The ET Tube cradle 14 includes a concavity 24 on one aspect, which, in the depicted embodiment, opens upwards or towards the patient's nose. This defines a seat on which the endotracheal tube may rest and be stabilized. The seat/concavity 24 may be equipped with ribs 26 to arrest motion of the ET Tube inwards or outwards by friction when the ET Tube is fixed in position.

The ET Tube is fixed in its position in the ET Tube cradle 14, in the depicted embodiment, by means of strap 28. Strap 28 may be fabricated of elastomeric material having some elasticity. The strap 28 is comprised of a thumb and finger tab 30, a stretchable ET Tube contact portion 32 and a through hole 34. In operation, after the tube stabilization device has been properly positioned and the ET Tube inserted into the airway to its proper depth, the strap 28 is stretched slightly outwards, wrapped over the top of the ET Tube on the ET Tube cradle 14 and then hole 34 is engaged with tab 36 to secure it in a locking position when released. The elasticity of the strap 28 serves to hold the hole 34 secure against the tab 36 and create pressure that holds the ET Tube securely in position against both lateral and axial movement relative to the ET Tube cradle 14.

In the depicted embodiment the support bar 12 may be fabricated of a malleable, semi-rigid material, for example brass or stainless steel, with an elastomeric over mold. Accordingly, metal rod 40 is covered with the elastomeric material 42.

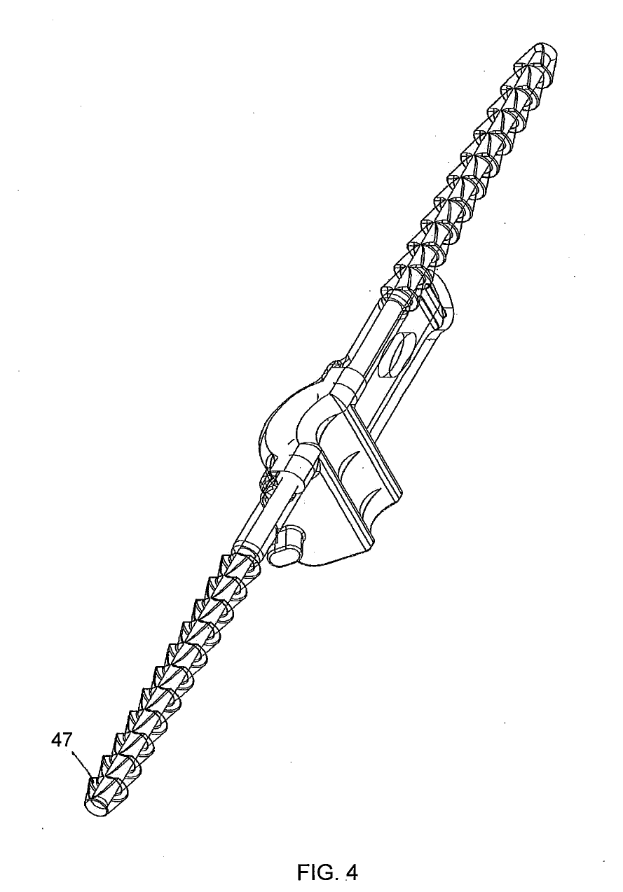

The support bar 12 is made of malleable material so that the left and right wings 44 may be bent around the mouth and cheeks of the patient. An originally straight and malleable configuration allows for ease of manufacturing and customizable shaping for each patient. The elastomeric material over the metal is configured to have at least one and, as depicted, a plurality of engagement elements 46. In the depicted embodiment these engagement elements 46 are teeth, which may be frustoconical. The depicted teeth are oriented with their narrow end outwards, to promote ease of assembly with the cheek pads 16. Of note and best seen in FIG. 4, the teeth are not fully circumferential around the stabilizing bar 40. This axially truncated shape 47 further promotes the proper orientation and positioning of the ET Tube cradle 14 relative to the patient's airway, due to the manner in which the flat side of the teeth engages with the corresponding structure in the cheek pads 16 as more fully described below.

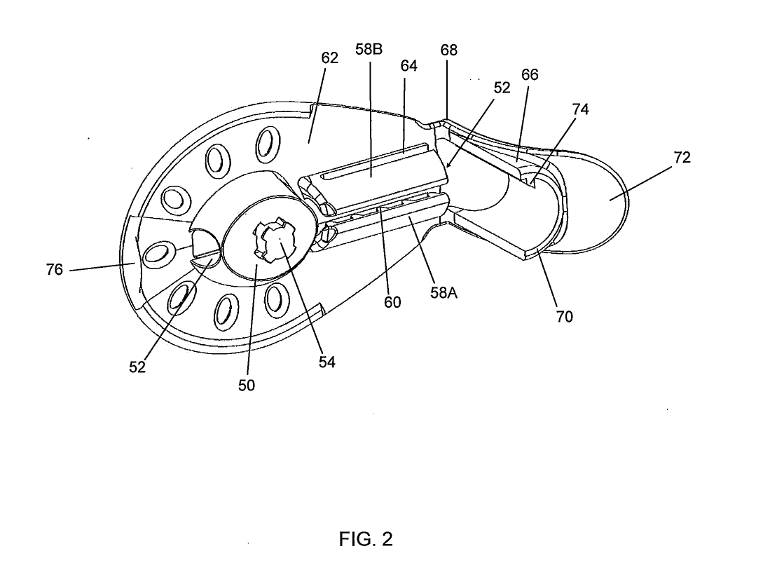

FIG. 2 depicts the cheek pad 16. An opposing side of the cheek pad away from view in FIG. 2 is substantially flat and configured to receive an interfacing material for contact with the patient's skin, such as an adhesive mole skin backing. The outwardly facing side of the cheek pad 16 includes the following components. A support bar receiving element 50 includes a passage 52 for receiving the support bar 12. In the depicted embodiment a neck strap post 54 is located on top of the receiving element 50. The neck strap post 54 is a positive element designed to engage the hole 56 at the end of the neck strap 18 (or any of the intermediate holes in the neck strap) for further tractive stabilization of the overall device. As the name implies, the neck strap 18 stretches from engagement of a left end hole 56 with a left cheek pad neck strap post 54 around the back of the patient's head to an opposing right side neck strap hole(s) 56 with the corresponding neck strap post 54 on the right cheek pad 16.

The bar retainer 58 defines the passage 52 into which the support bar 12 is inserted during assembly on the patient. In the depicted embodiment the receiver portions 58A and 58B includes teeth 60 configured and dimensioned to interlock with the spaces between the teeth or engagement elements 46 on the support bar 12. The teeth 60 on the cheek pad extend circumferentially around the inner surface of the retainer 58. However, in the depicted embodiment there are no ribs 60 on the surface of the outer planar surface 62 of the cheek pad that is located within the channel 52 for retaining the support bar. Hence the untoothed flat surface 62 matches with the flat lateral truncations 47 of the teeth or locking elements 46 in order to positively align the support bar 12 rotationally, which again promotes positioning of the ET Tube cradle 14 with the desired position aligned with the patient's airway. In the depicted embodiment the receiver 58 is bifurcated into portions 58a and 58b. Since the cheek pad 16 as depicted is molded of an elastomeric material, this gives the receiver 58 flexibility so that the teeth 60 may ride up and over the engaging elements 46 upon insertion of the support bar 12. Once the proper degree of insertion of the support bar 12 through the receiver 58 has been reached for an appropriate custom fit with a particular patient, the elasticity of the receiver wings 58 will bias them inwards towards a retaining, engaged position of teeth 60 with engaging elements 46. Receiver wings 58a and 58b, may have a thin portion molded into them, as represented by slot 64, to engage boss 74 on bar lock 66 and close channel 52 and secure bar 12.

Once the support bar 12 has been inserted into the receiver 58 of the cheek pad 16 to an appropriate position, that position may be locked into a holding engagement without further flexion of the receiver 58 by the use of the bar lock 66. Bar lock 66 is attached to the rest of the cheek pad 16 by a moveable element which, as depicted, may be a hinge 68. The bar lock 66 includes an encapsulating semicircle 70 dimensioned to fit over the outer surface of the receiver 58. A finger tab 72 facilitates its actuation by an operator. A boss 74 is dimensioned to engage slot 64 for positive locking into position. A symmetric cheek pad 16 is similarly assembled on the other side of the patient. Extension 76 serves as a finger tab and an end stop against over insertion of support bar 12.

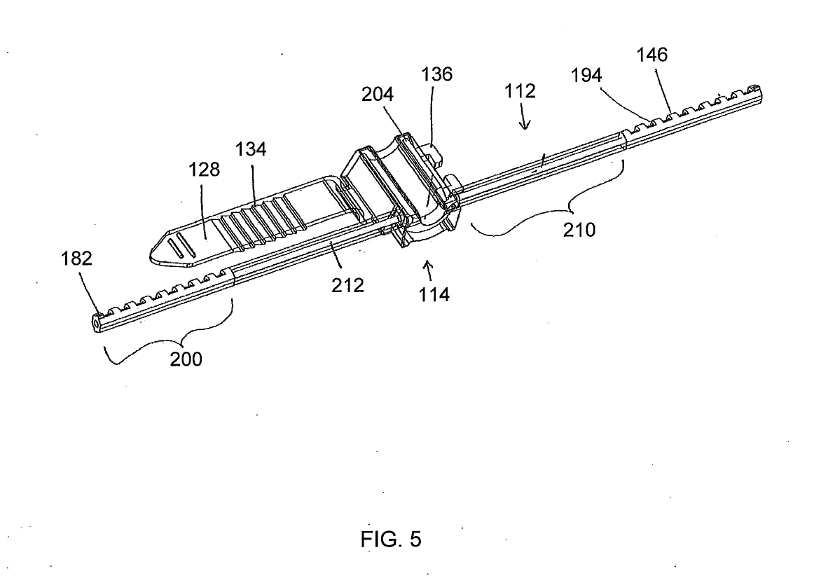

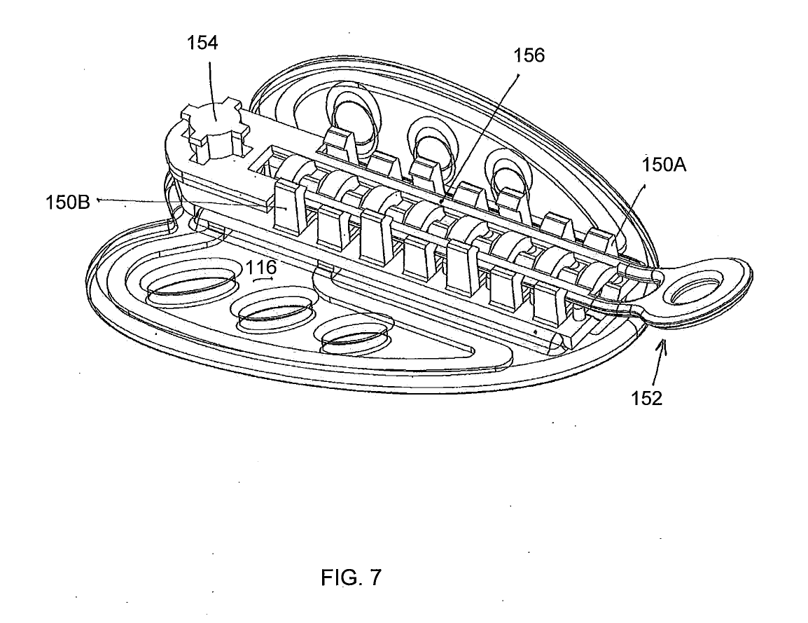

In an alternative embodiment, depicted in FIG. 5 the support bar 112 has alternative engagement elements 146. The cradle 114 and retaining strap 128 are substantially similar to the embodiment described above, with the exception that retaining means 134 comprise bosses for engagement in a receiving structure 136. The cheek pads 116 in the alternate embodiment include a plurality of hooked extensions 150 each comprised of a displaceable material such as plastic that is biased to return to and remain in a retaining position such as that depicted in FIG. 7. The retaining elements 150 are dimensioned to engage the retaining elements 146 on the rod 112.

The cheek pad 116 of this embodiment has three positions. In the engaged or closed position, those retaining elements 150 having the shorter vertical dimension 150A will retain a longitudinal locking element 152 in position to hold the stabilizer bar 112 on the cheek pad. Another position is completely removed from engagement, which position is provided for emergency removal of the bar 112 and cradle 114 apparatus. In such a circumstance, the longitudinal members 156 of the locking element 152 may be displaced inwardly for disengagement with the hooking elements of the retaining extensions 150, thereby making the entire locking element 152 removable from the cheek pad and thereafter, the rod 112 also removable therefrom. In an intermediate position, taller retaining elements 150B are used to temporarily engage the longitudinal elements 156 of the retaining strap 152. In this position, the stabilizer bar 112 may be translated through the channel 184 defined by the series of extension elements 150 in the cheek pad 116. This position is used for longitudinal adjustment the bar 112 and cheek pad 116 relative to one another, in order for the user to find an optimal position for the endotracheal stabilizer overall. Once the ideal position of the stabilizer bar 112 relative to the cheek pad 116 has been selected, the longitudinal elements 156 of the locking element 152 are pressed into the lower-most, retaining position to fix the user-selected position for ongoing placement and stabilization of an endotracheal tube. One end of locking element 152 extends beyond the check pad 116 as a tab to facilitate positioning or removal by being easier to grasp.

In the cheek pad assembly a post 178 is provided. In the depicted embodiment the post is somewhat conical and smooth. The outside dimension of post 178 matches the inner dimensions of post receiver 154 in retaining strap 152. The outer dimensions of post receiver 154 are used for mounting and seating a neck strap hole for adjustment of the neck strap. Any one of the adjustment holes in the neck strap may be placed over elements 54 or 154 for appropriate adjustment.

The smooth interface in the depicted embodiment between the outer surface of post 178 and the inner surface of post receiver 154 facilitates ease of vertical adjustment of the retaining strap 152 among its three different positions.

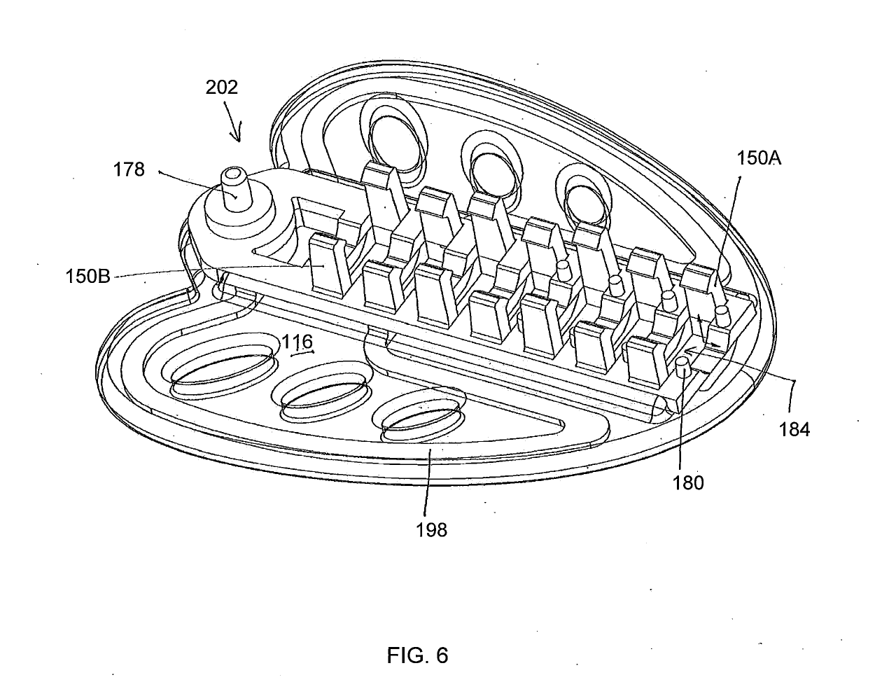

FIG. 6 further depicts retaining posts 180 that project upwardly into a channel defined for receiving the stabilizing bar 112. After assembly, the innermost of these posts 180—that is the post 180 most distal from the patient's mouth—will engage tabs 182 in an end area of the stabilizer bar 112. By abutting the retaining posts 180, the tabs 182 arrest separation or axial movement of the stabilizer bar 112 outwards from the channel 184 defined for it in the cheek pad receiver assembly.

To facilitate sanitary packaging and ease of application of the device on the patient, the nurse receives the stabilizer bar and cheek pad in a pre-assembled position within a sealed package which may optionally be sterile. The cheek pad/stabilizer bar assembly is packaged in the intermediate position, ready for adjustment to the individual patient as described above, but not needing to be assembled together. The tube holder device is also removed from its sealed packaging with a cover over the adhesive surface on the inside of the cheek pads. The adjustment procedure allows for adjustments to be made and the stabilizer bar, retaining strap and cheek pad receiver assembly to be placed in their lockdown position before removal of the adhesive cover and application of the adhesive inner surface of the cheek pad to the patient.

Upon pressure downwardly on the retaining strap 152 in order to place the longitudinal elements 156 in the lockdown position, the outwardly facing bosses 146 of the stabilizer bar 112 will engage the rungs or transverse members 192 of the retaining strap 152. Hence, the stabilizer bar 112 is restrained from being withdrawn improperly from the cheek pad by the abutment of a proximal side 194 of each boss 146 with a distal side 196 of the transverse members 192 of the retaining strap 152. As a further assurance against improper withdrawal, the tabs 182 would be arrested from improper withdrawal by pegs 180 in any event. Before the longitudinal elements 156 of the retaining strap 152 are placed in their lockdown position, the tabs 182 do not interfere with longitudinal travel of the stabilizer bar 112 in the receiving channel 184.

The overall cheek pad assembly flexes and is bendable to facilitate a comfortable adjustment and placement on the patient in a manner that enables the device to be contoured to the patient's facial features. An appropriate amount of rigidity is maintained by a stiffer skeleton element 198 within the cheek pad.

The cradle or sled for receiving the endotrachael tube in the depicted embodiments would have a friction surface for arresting undesirable travel of the endotrachael tube in or out of the patient's mouth. This may be by means of the ribs 26 depicted in the first embodiment or, in the alternative, may include, without limitation a soft elastomeric material having a higher coefficient of friction to serve the same purpose.

The present invention also includes an advantageous method of manufacture. The depicted embodiments are comprised of a malleable metal stabilizing rod core over which the various operational elements and features of the invention are molded in polymers. A harder polymer such as, for example without limitation, polyurethane, may be used where greater rigidity may be advantageous, for example the outer sections of the stabilizing rod 112 such as at area 200 having the teeth 46/146 and in the stabilizer bar receiver assembly 202 on the cheek pads 116. The same is true for inner components 204 of the tube cradle. The same may be true for tube strap receiver 136. Other portions of the device of the present invention may be more advantageously made of a soft material such as a more flexible elastomeric, including for example portion 210 of the stabilizer bar and strap 128.

According to the method of manufacturing the present invention, the rigid but malleable rod 212 would be placed in a first mold or a first concave of a single mold having a first insert that would block or fill the space to be later filled by the softer elastomeric material. The tool would then define as its inner concavity the shape of the harder plastic elements 200 (or 202). These would be injected and molded. After cooling and extraction of the stabilizer rod core 212 with the hard plastic components now attached, the work piece is placed in a second mold, or a second portion of the same mold, having a different internal concavity. The work piece assembly 212/200 is put in position in order to receive the injection of the softer elastomeric material, in order to fabricate portions 210 and the other soft polymer portions of the device. Advantageously, the structure of the bosses 146, tabs 182 of hard plastic in the portions 200 of the stabilizing rod 112 may be used to hold, stabilize and/or center the work piece in the mold for injection of the softer elastomeric material. Hence by over molding the rod 212 is properly positioned in the softer elastomeric material.

As various modifications could be made to the exemplary embodiments, as described above with reference to the corresponding illustrations, without departing from the scope of the invention, it is intended that all matter contained in the foregoing description and shown in the accompanying drawings shall be interpreted as illustrative rather than limiting. Thus, the breadth and scope of the present invention should not be limited by any of the above-described exemplary embodiments, but should be defined only in accordance with the following claims appended hereto and their equivalents.

What is claimed is:

1. An endotracheal tube stabilizer comprising:
a tube cradle;
a tube fixation element attached to said tube cradle;
a stabilization bar having a plurality of engagement elements, said stabilization bar extending on either side of said tube cradle;
at least one cheek pad having a releasable engagement interface dimensioned to engage said engagement elements of said stabilization rod in at least one engaged position.

2. The endotracheal tube stabilizer claim 1 wherein said endotracheal tube stabilizer is a neonatal endotracheal tube stabilizer.

3. The endotracheal tube stabilizer of claim 1 wherein said cheek pad further comprises an adjustment position, said adjustment position being configured to provide user selectable longitudinal adjustment of said bar relative to said cheek pad.

4. The endotracheal tube stabilizer of claim 3 wherein said engagement interface of said cheek pad further comprises a locking element and a plurality of extensions to engage said locking element such that in a first adjustable position, user selectable longitudinal movement of said bar through a channel defined by said plurality of extensions is provided and said extensions further engaging said bar in a locked position by retention of said locking element in a locked position, said locked position retaining said bar in a nonmoveable state.

5. The endotracheal tube stabilizer of claim 1 wherein said engagement elements of said stabilization bar are teeth dimensioned to engage transverse members of a cheek pad assembly.

6. The endotracheal tube stabilizer of claim 5 wherein said transverse members are on a locking element.

7. The endotracheal tube stabilizer of claim 1 wherein said stabilization bar engagement elements are teeth, said teeth being flat on one side.

8. The endotracheal tube stabilizer of claim 4 wherein a first plurality of extensions has a first length, said first plurality of extensions being configured to retain said locking element and said bar in a locked position and wherein said engagement interface of said cheek pad has a second plurality of extensions having a second length, said second length being configured to constrain said locking element and said bar in said adjustable position.

9. The endotracheal tube stabilizer of claim 1 wherein said engagement interface of said cheek pad further comprises an emergency release assembly.

10. The endotracheal tube stabilizer of claim 1 wherein said stabilization bar and said tube cradle are integrally formed of a malleable metallic rod, a first more rigid polymer comprising said tube cradle and a second less rigid polymer comprising at least one of said engagement elements of said stabilization bar, said engagement interface of said cheek pad and said tube fixation element.

11. The endotracheal tube stabilizer of claim 1 further comprising a package in which said endotracheal tube stabilizer is provided to a user already in a longitudinally adjustable engagement position between said stabilization bar'and said cheek pad.

12. The endotracheal tube stabilizer of claim 1 wherein said tube fixation element is a strap and a lock.

13. A method of manufacturing an endotracheal tube stabilizer comprising:
providing a malleable rod;
forming a tube cradle on said rod;
attaching a tube fixation element on one of said tube cradle or said rod;
providing a cheek pad;
attaching a releasable engagement interface to at least one of said cheek pad or said rod such that said rod may be longitudinally adjusted relative to said cheek pad in a user selectable position and such that after said adjustment, said rod and said cheek pad may be locked in said user selected position.

14. The method of claim 13 further comprising forming said tube cradle of a first more rigid polymer and forming from a less rigid polymer at least one of said tube fixation element or said rod engagement elements.

15. The method of claim 13 wherein said step of forming said tube cradle of said more rigid polymer is performed first and said step of forming said tube fixation element or said engagement elements of said second less rigid polymer is performed second.

16. The method of claim 13 further comprising a step of providing a first plurality of cheek pad extensions having a first length dimensioned to define an adjustment channel for said rod and said rod engagement elements and further providing a second plurality of extensions on said cheek pad, said second plurality of extensions defining a locking engagement with said engagement elements of said rod.

17. The method of claim 13 further comprising attaching a locking element on said cheek pad, said locking element being dimensioned and positioned to mediate said adjustment position and said locking position of said stabilization rod relative to said cheek pad.

18. The method of claim 13 further comprising providing a neck strap with said endotracheal tube stabilizer.

19. The method of claim 13 further comprising providing an adhesive backing for said cheek pad.
